(19) United States
(12) Patent Application Publication
(10) Pub. No.: US 2026/0345655 A1
(43) Pub. Date: Nov. 5, 2026

(54) Inhibiting Purine Biosynthesis to Increase Favipiravir Potency Against Orthohantavirus Infections

(71) Applicant: Viriom, Inc., Rockville, MD (US)

(72) Inventors: Charles David Pauza, Gaithersburg, MD (US); Nikolay Savchuk, San Diego, CA (US)

(21) Appl. No.: 19/705,789

(22) Filed: Jun. 12, 2026

Related U.S. Application Data
(63) Continuation-in-part of application No. 18/866,525, filed on May 13, 2023.
(60) Provisional application No. 63/342,335, filed on May 16, 2022.

(51) Int. Cl. A61K 31/706; A61K 31/365; A61K 31/4965; A61K 31/5377; A61P 31/16

(52) U.S. Cl. CPC A61K 31/706; A61K 31/365; A61K 31/4965; A61K 31/5377; A61P 31/16

(57) ABSTRACT

The present invention is generally directed to methods for the treatment and prophylaxis of Orthohantavirus infections that involve combinations of therapeutic agents. Described herein are combinations of two or more drugs, wherein the combination inhibits Orthohantavirus replication through one or more mechanisms of action and enhances the antiviral activity of nucleoside and nucleotide analog compounds through inhibition of cellular enzymes involved in purine nucleotide biosynthesis. The combinations may be administered sequentially, concurrently, or as co-formulated pharmaceutical compositions comprising two or more agents. The inventive aspects include the identification of combination components, ratios among the components, and treatment regimens for reducing morbidity and mortality associated with Orthohantavirus infection. Further provided are methods of administration and delivery of the disclosed combinations.

### CROSS-REFERENCE TO RELATED APPLICATIONS

This application is a continuation-in-part of U.S. patent application Ser. No. 18/866,525, filed Nov. 15, 2024, and entitled “Inhibiting Purine Biosynthesis to Increase Favipiravir Potency Against RNA Virus Infections,” which is the national stage of International Patent Application No. PCT/US2023/066978, filed May 13, 2023, and entitled “Inhibiting Purine Biosynthesis to Increase Favipiravir Potency Against RNA Virus Infections,” which claims the benefit of and priority to U.S. Provisional Patent Application No. 63/342,335, filed May 16, 2022, and entitled “Inhibiting Purine Biosynthesis to Increase Favipiravir Potency Against RNA Virus Infections.” The disclosures of the foregoing applications are hereby incorporated herein by reference in their entirety for all purposes.

### FIELD OF INVENTION

The present invention relates to the field of antiviral therapy and, more particularly, to methods and pharmaceutical compositions for the treatment and prevention of infections caused by Orthohantavirus.

### BACKGROUND

Orthohantaviruses are enveloped, negative-sense RNA viruses belonging to the family Hantaviridae and are responsible for significant human disease worldwide. Human infection typically occurs through inhalation of aerosolized excreta from infected rodent hosts. Several Orthohantavirus species are associated with severe disease in humans, including Hantavirus Cardiopulmonary Syndrome (HCPS), also known as Hantavirus Pulmonary Syndrome (HPS), and Hemorrhagic Fever with Renal Syndrome (HFRS). Mortality rates associated with certain Orthohantavirus infections may exceed 30%, highlighting the need for effective antiviral interventions.

Orthohantavirus infections remain a substantial public health concern in North America, South America, Europe, and Asia. Numerous outbreaks have been reported involving Sin Nombre virus, Andes virus, Hantaan virus, Dobrava-Belgrade virus, and Seoul virus. Despite extensive research efforts, therapeutic options for Orthohantavirus infections remain limited, and no broadly effective antiviral treatment has been established for routine clinical use in many jurisdictions.

Orthohantaviruses replicate using a virally encoded RNA-dependent RNA polymerase (RdRp), which is responsible for synthesis of viral genomic RNA, antigenomic RNA, and messenger RNA. Viral RNA replication is essential for production of infectious viral particles and therefore represents an attractive target for antiviral intervention. Inhibition of viral RNA synthesis can reduce viral replication, suppress viral load, and limit disease progression.

Several antiviral compounds have demonstrated activity against Orthohantaviruses in cell culture, animal models, or limited clinical studies. Ribavirin has shown efficacy against certain Orthohantavirus species when administered early in infection. Favipiravir has demonstrated broad-spectrum antiviral activity against numerous RNA viruses and has shown inhibitory activity against Orthohantaviruses in experimental models.

Favipiravir is converted intracellularly to its active ribofuranosyl triphosphate metabolite and competes with naturally occurring purine nucleotides during viral RNA synthesis. Incorporation of the active metabolite into nascent viral RNA may impair replication fidelity, increase mutation frequency, and reduce viral fitness. The antiviral activity of Favipiravir may be influenced by intracellular nucleotide pools, particularly guanosine nucleotides that compete with the active metabolite for incorporation by the viral polymerase.

The antiviral potency of nucleoside analog compounds may therefore be enhanced by reducing intracellular concentrations of competing endogenous nucleotides. Inhibition of selected enzymes involved in purine biosynthesis can alter nucleotide pool composition and increase the likelihood of antiviral nucleotide analog incorporation into viral RNA.

Various inhibitors of de novo purine biosynthesis have been developed for treatment of viral infections, autoimmune disorders, transplantation-related conditions, and malignancies. Examples include inhibitors of inosine monophosphate dehydrogenase (IMPDH), such as mycophenolic acid, which reduce intracellular guanosine nucleotide pools and exhibit antiviral activity against numerous RNA viruses.

Although antiviral nucleoside analogs and purine biosynthesis inhibitors have been studied individually, there remains a need for methods capable of more effectively inhibiting Orthohantavirus replication. Rationally designed combinations targeting both viral RNA synthesis and host nucleotide biosynthesis pathways may provide enhanced antiviral activity compared with either approach alone.

The present invention provides methods for inhibiting replication of Orthohantaviruses by administering one or more antiviral nucleoside analogs in combination with one or more inhibitors of purine biosynthesis. The invention further provides pharmaceutical compositions, dosing regimens, and combinations that exhibit enhanced inhibition of Orthohantavirus replication while maintaining acceptable safety profiles.

### SUMMARY

A first aspect of the invention provides a method for treating a disease or disorder associated with an RNA virus infection, the method comprising administering to a patient in need thereof a therapeutically effective amount of (i) favipiravir in combination with (ii) mycophenolate.

Another aspect of the invention provides a method for treating a disease or disorder associated with an Orthohantavirus infection, the method comprising administering to a patient in need thereof a therapeutically effective amount of a combination product or pharmaceutical composition described herein.

Another aspect of the invention is directed to a method of inhibiting RNA virus replication by inhibiting purine biosynthesis. The method involves administering to a patient in need thereof an effective amount of a combination product or a pharmaceutical composition described herein.

Another aspect of the invention is directed to a method of inhibiting Orthohantavirus replication coupled with inhibiting purine biosynthesis. The method involves administering to a patient in need thereof an effective amount of a combination product or a pharmaceutical composition described herein.

Another aspect of the invention is directed to a method of treating or preventing a disease or disorder disclosed herein in a subject in need thereof. The method involves administering to a patient in need of the treatment an effective amount of a combination product or a pharmaceutical composition disclosed herein.

Another aspect of the present invention relates to the use of a combination product or a pharmaceutical composition thereof, in the treatment of a disease or disorder disclosed herein.

The present invention further provides methods of treating a disease or disorder associated with viral infection, comprising administering to a patient suffering from at least one of said viral infection a combination product or pharmaceutical composition thereof.

The present invention further provides methods of preventing, treating, or ameliorating a viral infection caused by virus selected from Andes hantavirus (ANDV), Sin Nombre Hantavirus (SNV), Four Corners hantavirus (SNV), Seoul virus (SEOV), Laguna Negra virus (LANV), Araraquara virus, Juquitiba virus, Dobrava virus (DOBV), Puumala virus (PUUV), Black Creek Canal virus, Choclo virus, Lechiguanas virus, Monongahela virus, New York virus, Tula virus, Amur virus, Sangassou virus, Thottapalayam virus (TPMV), Oxbow virus, Luxi virus and new hantaviruses that may be discovered or viruses that may be renamed.

The present invention further provides methods of treating, or ameliorating a viral infection caused by virus selected from Andes hantavirus (ANDV), Sin Nombre Hantavirus (SNV), Four Corners hantavirus (SNV), Seoul virus (SEOV), Laguna Negra virus (LANV), Araraquara virus, Juquitiba virus, Dobrava virus (DOBV), Puumala virus (PUUV), Black Creek Canal virus, Choclo virus, Lechiguanas virus, Monongahela virus, New York virus, Tula virus, Amur virus, Sangassou virus, Thottapalayam virus (TPMV), Oxbow virus, Luxi virus and new hantaviruses that may be discovered or viruses that may be renamed comprising administering to a patient suffering from at least one of said viral infections a therapeutically effective amount of favipiravir in combination with mycophenolate.

In some aspects, the present disclosure provides a method of preparing a combination product or a pharmaceutical composition of the present disclosure.

In some aspects, the present disclosure provides a method of preparing combination product of the present disclosure, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

The present disclosure provides methods of treating, preventing, or ameliorating a disease or disorder in which associated with Orthohantavirus infection by administering to a subject in need thereof a therapeutically effective amount of a combination product as disclosed herein.

The inventors found that favipiravir in combination with mycophenolate show therapeutic activities that are useful in therapy, in particular for the treatment of infections caused by Orthohantavirus.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### DEFINITIONS

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “combination” and/or “combination product” refers to a product composed of any combination of a drug. Each drug included in a combination product is referred to as a “constituent part” of the combination product. The combination product is a kit of parts comprising components i) and ii), for sequential, separate, or simultaneous use or a composition (a mixture) of two components i) and ii).

The term “pharmaceutical composition” refers to admixture of different chemical substances, including the active drug (or number of active drugs), are combined to produce a final medicinal product.

A “patient” or “subject” is a mammal, e.g., a human, mouse, rat, guinea pig, dog, cat, horse, cow, pig, or non-human primate, such as a monkey, chimpanzee, baboon, or rhesus.

An “effective amount” when used in connection with a compound is an amount effective for treating or preventing a disease in a subject as described herein.

The term “carrier”, as used in this disclosure, encompasses carriers, excipients, and diluents, and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

A “viral disease” (or “viral infection”, abbreviated vid or VID) occurs when an organism's body is invaded by pathogenic viruses, and infectious virus particles (virions) attach to and enter susceptible cells.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed combination product or a composition to a subject, or administering a prodrug derivative or analog of the compounds of combination product or composition to the subject, which can form an equivalent amount of active compound within the subject's body.

The term “prodrug”, as used in this disclosure, means a compound which is convertible in vivo by metabolic means (e.g., by hydrolysis) to a disclosed compound.

The term “salt” refers to pharmaceutically acceptable salts.

“Inhibitors RNA virus replication” as used herein refer to combination product and/or compositions comprising a combination product which inhibit of viral RNA replication.

The amount of combination product or composition described herein needed for achieving a therapeutic effect may be determined empirically in accordance with conventional procedures for the particular purpose. Generally, for administering therapeutic agents (e.g. combination products or compositions (and/or additional agents) described herein) for therapeutic purposes, the therapeutic agents are given at a pharmacologically effective dose.

A “pharmacologically effective amount”, “pharmacologically effective dose”, “therapeutically effective amount”, or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. For example, administration of therapeutic agents to a subject suffering from Orthohantavirus infection provides a therapeutic benefit not only when the underlying condition is eradicated or ameliorated, but also when the subject reports a decrease in the severity or duration of the symptoms associated with the disease, e.g., a decrease in viral burden, a decrease in circulating of Orthohataviruses, an increase in progression free survival. Therapeutic benefit also includes halting or slowing the progression of the underlying disease or disorder, regardless of whether improvement is realized.

### Combination Product of the Present Disclosure

The combination product according to invention comprises:
(i) favipiravir and
(ii) mycophenolate.

In some embodiments, the combination product comprises favipiravir (i) as a nucleoside base (favipiravir), favipiravir ribonucleotide or favipiravir nucleotide triphosphate (favipiravir-NTP):

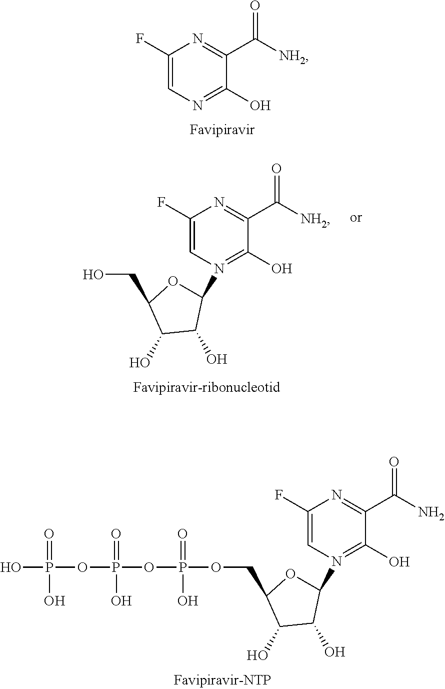

or a pharmaceutically acceptable salt, solvate, or tautomer thereof.

In some embodiments, the combination product comprises favipiravir (i) as a nucleoside base (favipiravir).

In some embodiments, the combination product comprises favipiravir (i) as a favipiravir ribonucleotide.

In some embodiments, the combination product comprises favipiravir (i) as a favipiravir nucleotide triphosphate (favipiravir-NTP).

Favipiravir, a purine nucleoside analogue capable of causing lethal mutagenesis during RNA virus replication.

In some embodiments, the combination product comprises mycophenolate (ii) as a mycophenolic acid, sodium salt of mycophenolic acid, or mycophenolate mofetil:

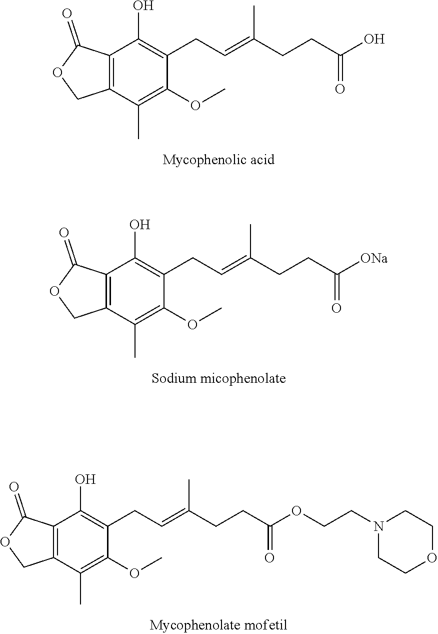

In some embodiments, the combination product comprises mycophenolate (ii) as a mycophenolic acid.

In some embodiments, the combination product comprises mycophenolate (ii) as a sodium salt of mycophenolic acid.

In some embodiments, the combination product comprises mycophenolate (ii) as a mycophenolate mofetil.

In some embodiments, the combination product is selected from the combinations described in Table 1.

TABLE 1
Examples of composition of combination product.

| | Favipiravir | Mycophenolate | Molar ratio |
|---|---|---|---|
| # | (i) | (ii) | (i)/(ii) |
| 1 | favipiravir | mycophenolic acid | 1:100 - 100:1 |
| 2 | favipiravir ribonucleotide | mycophenolic acid | 1:100 - 100:1 |
| 3 | favipiravir-NTF | mycophenolic acid | 1:100 - 100:1 |
| 4 | favipiravir | sodium mycophenolate | 1:100 - 100:1 |
| 5 | favipiravir ribonucleotide | sodium mycophenolate | 1:100 - 100:1 |
| 6 | favipiravir-NTF | sodium mycophenolate | 1:100 - 100:1 |
| 7 | favipiravir | mycophenolate mofetil | 1:100 - 100:1 |
| 8 | favipiravir ribonucleotide | mycophenolate mofetil | 1:100 - 100:1 |
| 9 | favipiravir-NTF | mycophenolate mofetil | 1:100 - 100:1 |

In some embodiments, compounds formed combination product described herein are pharmaceutically acceptable salts, stereoisomers, solvates, prodrugs, isotopic derivatives, or tautomers of these compounds.

A suitable pharmaceutically acceptable salt of a compound of the combination is, for example, an acid-addition salt of a compound of the disclosure, which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It would be understood that the compounds of any one of the combination products disclosed herein and any pharmaceutically acceptable salts thereof, comprise stereoisomers, mixtures of stereoisomers, polymorphs of all isomeric forms of said compounds.

In some embodiments, the isotopic derivative is a deuterium labeled compound.

The term “isotopic derivative”, as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I). In some embodiments, the isotopic derivative is enriched with regard to, or labelled with, one or more atoms selected from ²H, ¹³C, ¹⁴C, ¹⁵N, and ¹⁸O. In some embodiments, the isotopic derivative is a deuterium labeled compound (i.e., being enriched with ²H with regard to one or more atoms thereof).

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is to be understood that the compounds of combination product of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

The compounds of combination product of this disclosure may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E- and Z-isomers). It is to be understood that the present disclosure encompasses all optical, diastereoisomers and geometric isomers and mixtures thereof that possess inflammasome inhibitory activity.

It is also to be understood that certain compounds of any one of combination product disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the combination products disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

The compounds of any one of the combination products disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the compounds of combination products disclosed herein.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the combination products disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl) piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the combination products disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the combination products disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl,morpholinomethyl,piperazin-1-ylmethyl and 4-(C₁-C₄ alkyl) piperazin-1-ylmethyl.

In some embodiments, the combination product is a combination of a pharmaceutical composition of favipiravir (i) and a pharmaceutical composition of mycophenolate (ii).

In some embodiments, the combination product is a kit of parts comprising components (i) and (ii), for sequential, separate, or simultaneous use.

In some embodiments, the combination product is a kit of parts comprising components (i) and (ii) for sequential use.

In some embodiments, the combination product is a kit of parts comprising components (i) and (ii) for separate use.

In some embodiments, the combination product is a kit of parts comprising components (i) and (ii) for simultaneous use.

In some embodiments, the combination product is a composition comprising components (i) and (ii) and a pharmaceutically acceptable carrier.

The in vivo effects of a compound of any one of the combination products disclosed herein may be exerted in part by one or more metabolites that are formed within the human or animal body after administration of a compound of any one of the combination products disclosed herein. As stated hereinbefore, the in vivo effects of a compound of any one of the combination products disclosed herein may also be exerted by way of metabolism of a precursor compound (a prodrug).

### Pharmaceutical Compositions

In some aspects, the present disclosure provides a pharmaceutical composition comprising the compounds of the combination product as active ingredients. In some embodiments, the present disclosure provides a pharmaceutical composition comprising favipiravir (i) and mycophenolate (ii), or their pharmaceutically acceptable salt or solvate, and one or more pharmaceutically acceptable carriers or excipients.

In some embodiments, the present disclosure provides a pharmaceutical composition comprising (i) favipiravir or pharmaceutically acceptable salt, solvate, or tautomer thereof; (ii) mycophenolate or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof; (ii) mycophenolic acid (MPA) or pharmaceutically acceptable salt, solvate, or tautomer thereof and a pharmaceutically acceptable carrier.

In some embodiments, a pharmaceutical composition comprising (i) favipiravir or pharmaceutically acceptable salt, solvate, or tautomer thereof; (ii) mycophenolate mofetil or pharmaceutically acceptable salt, solvate, or tautomer thereof and a pharmaceutically acceptable carrier.

As used herein, the term “pharmaceutical composition” is intended to encompass a product comprising the specified ingredients in the specified amounts, as well as any product which results, directly or indirectly, from combination of the specified ingredients in the specified amounts.

The pharmaceutical composition of present disclosure can be formulated for oral administration in forms such as tablets, capsules (each of which includes sustained release or timed-release formulations), pills, powders, granules, elixirs, tinctures, suspensions, syrups, and emulsions.

In some embodiments, a pharmaceutical composition of present disclosure can be formulated for oral administration in form of tablets.

In some embodiments, a pharmaceutical composition of present disclosure can be formulated for oral administration in form of capsules (each of which includes sustained release or timed-release formulations).

In some embodiments, a pharmaceutical composition of present disclosure can be formulated for oral administration in form of pills.

In some embodiments, a pharmaceutical composition of present disclosure can be formulated for oral administration in form of powders.

In some embodiments, a pharmaceutical composition of present disclosure can be formulated for oral administration in form of granules.

In some embodiments, a pharmaceutical composition of present disclosure can be formulated for oral administration in form of elixirs.

In some embodiments, a pharmaceutical composition of present disclosure can be formulated for oral administration in form of tinctures.

In some embodiments, a pharmaceutical composition of present disclosure can be formulated for oral administration in form of suspensions.

In some embodiments, a pharmaceutical composition of present disclosure can be formulated for oral administration in form of syrups.

In some embodiments, a pharmaceutical composition of present disclosure can be formulated for oral administration in form of emulsions.

The pharmaceutical composition of present disclosure can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio) propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetylpyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

In some embodiments, examples of a preservative include those selected from the group consisting of quaternary ammonium salts such as benzalkonium halides (preferably benzalkonium chloride), chlorhexidine gluconate, benzethonium chloride, cetylpyridinium chloride, benzyl bromide, phenylmercury nitrate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. In some embodiments, the tonicity agent is selected from the group consisting of a glycol (such as propylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols-such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base-depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises compounds (i) and (ii) of a combination product of the disclosure as defined hereinbefore, or a pharmaceutically acceptable salt, hydrate or solvate thereof, in association with a pharmaceutically acceptable diluent or carrier.

In some embodiments, a pharmaceutical composition described herein may further comprise one or more additional pharmaceutically active agents.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The pharmaceutical compositions of the disclosure may be obtained by conventional procedures using conventional pharmaceutical excipients, well known in the art. Thus, compositions intended for oral use may contain, for example, one or more coloring, sweetening, flavoring and/or preservative agents.

A therapeutically effective amount of a compound of the present disclosure for use in therapy is an amount sufficient to treat or prevent a viral infection referred to herein, slow its progression and/or reduce the symptoms associated with the condition.

A therapeutically effective amount of a compound of the present disclosure for use in therapy is an amount sufficient to treat a viral infection related condition referred to herein, slow its progression and/or reduce the symptoms associated with the condition.

The size of the dose for therapeutic or prophylactic purposes of a combination product will naturally vary according to the nature and severity of the conditions, the age and sex of the animal or subject and the route of administration, according to well-known principles of medicine.

### Biological Assays

Combination product designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the combination products described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the combination products of the present disclosure. These in vitro or in vivo biological assays can include, but are not limited to, enzymatic activity assays, electrophoretic mobility shift assays, reporter gene assays, in vitro cell viability assays, and the assays described herein.

### Methods of Use

In some aspects, the present disclosure provides a method of inhibition of replication of RNA virus (e.g., in vitro or in vivo), comprising contacting a cell with a therapeutically effective amount of a combination product of the present disclosure.

In some aspects, the present disclosure provides a method of treating or preventing a disease or disorder disclosed herein in a subject in need thereof, comprising administering to the subject a therapeutically effective amount of a combination product of the present disclosure or a pharmaceutical composition of the present disclosure.

In some aspects, the present disclosure provides a method of treating a disease or disorder disclosed herein in a subject in need thereof, comprising administering to the subject a therapeutically effective amount of a combination product of the present disclosure or a pharmaceutical composition of the present disclosure.

In some embodiments, the disease or disorder is an RNA viral infection.

In some embodiments, the disease or disorder is a disease or disorder in which RNA viral infection is implicated.

In some embodiments, the disease or disorder is an Orthohantavirus infection.

In some embodiments, the disease or disorder is a disease or disorder in which Orthohantavirus infection is implicated.

The combination product of the invention is an antiviral agent.

The pharmaceutical composition of the invention is an antiviral agent.

In some embodiments, the combination product, pharmaceutical compositions, and methods disclosed herein are used in the prevention or treatment of a viral infection.

The combination product of the invention is also useful in treating viral infections caused by Orthohantavirus. For example, viral infections treatable according to the methods of the invention caused by from Andes hantavirus (ANDV), Sin Nombre Hantavirus (SNV), Four Corners hantavirus (SNV), Seoul virus (SEOV), Laguna Negra virus (LANV), Araraquara virus, Juquitiba virus, Dobrava virus (DOBV), Puumala virus (PUUV), Black Creek Canal virus, Choclo virus, Lechiguanas virus, Monongahela virus, New York virus, Tula virus, Amur virus, Sangassou virus, Thottapalayam virus (TPMV), Oxbow virus, and Luxi virus.

In some embodiments, the viral infection is Andes hantavirus (ANDV) infection.

In some embodiments, the viral infection is Sin Nombre Hantavirus (SNV) infection.

In some embodiments, the viral infection is Four Corners hantavirus (SNV) infection.

In some embodiments, the viral infection is Seoul virus (SEOV) infection.

In some embodiments, the viral infection is Laguna Negra virus (LANV) infection.

In some embodiments, the viral infection is Araraquara virus infection.

In some embodiments, the viral infection is Juquitiba virus infection.

In some embodiments, the viral infection is Dobrava virus (DOBV) infection.

In some embodiments, the viral infection is Puumala virus (PUUV) infection.

In some embodiments, the viral infection is Black Creek Canal virus infection.

In some embodiments, the viral infection is Choclo virus infection.

In some embodiments, the viral infection is Lechiguanas virus infection.

In some embodiments, the viral infection is Monongahela virus infection.

In some embodiments, the viral infection is New York virus infection.

In some embodiments, the viral infection is Tula virus infection.

In some embodiments, the viral infection is Amur virus infection.

In some embodiments, the viral infection is Sangassou virus infection.

In some embodiments, the viral infection is Thottapalayam virus (TPMV) infection.

In some embodiments, the viral infection is Oxbow virus infection.

In some embodiments, the viral infection is Luxi virus infection.

In some aspects, the present disclosure provides a method of treating or preventing an Andes hantavirus (ANDV) infection in a subject in need thereof, comprising administering to the subject a therapeutically effective amount of a combination product of the present disclosure or a pharmaceutical composition of the present disclosure.

In some aspects, the present disclosure provides a method of treating or preventing a Sin Nombre Hantavirus (SNV) infection in a subject in need thereof, comprising administering to the subject a therapeutically effective amount of a combination product of the present disclosure or a pharmaceutical composition of the present disclosure.

In some aspects, the present disclosure provides a method of treating or preventing a Four Corners hantavirus (SNV) infection in a subject in need thereof, comprising administering to the subject a therapeutically effective amount of a combination product of the present disclosure or a pharmaceutical composition of the present disclosure.

In some aspects, the present disclosure provides a method of treating or preventing a Seoul virus (SEOV) infection in a subject in need thereof, comprising administering to the subject a therapeutically effective amount of a combination product of the present disclosure or a pharmaceutical composition of the present disclosure.

In some aspects, the present disclosure provides a method of treating or preventing a Laguna Negra virus (LANV) infection in a subject in need thereof, comprising administering to the subject a therapeutically effective amount of a combination product of the present disclosure or a pharmaceutical composition of the present disclosure.

In some aspects, the present disclosure provides a method of treating or preventing an Araraquara virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Juquitiba virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Dobrava virus (DOBV) infection in a subject in need thereof, comprising administering to the subject a therapeutically effective amount of a combination product of the present disclosure or a pharmaceutical composition of the present disclosure.

In some aspects, the present disclosure provides a method of treating or preventing a Puumala virus (PUUV) infection in a subject in need thereof, comprising administering to the subject a therapeutically effective amount of a combination product of the present disclosure or a pharmaceutical composition of the present disclosure.

In some aspects, the present disclosure provides a method of treating or preventing a Black Creek Canal virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Choclo virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Lechiguanas virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Monongahela virus infection in a subject in need thereof, comprising administering to the subject a therapeutically effective amount of a combination product of the present disclosure or a pharmaceutical composition of the present disclosure.

In some aspects, the present disclosure provides a method of treating or preventing a New York virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Tula virus infection in a subject in need thereof, comprising administering to the subject a therapeutically effective amount of a combination product of the present disclosure or a pharmaceutical composition of the present disclosure.

In some aspects, the present disclosure provides a method of treating or preventing an Amur virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Sangassou virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Thottapalayam virus (TPMV) infection in a subject in need thereof, comprising administering to the subject a therapeutically effective amount of a combination product of the present disclosure or a pharmaceutical composition of the present disclosure.

In some aspects, the present disclosure provides a method of treating or preventing an Oxbow virus infection in a subject in need thereof, comprising administering to the subject a therapeutically effective amount of a combination product of the present disclosure or a pharmaceutical composition of the present disclosure.

In some aspects, the present disclosure provides a method of treating or preventing a Luxi virus infection in a subject in need thereof, comprising administering to the subject a therapeutically effective amount of a combination product of the present disclosure or a pharmaceutical composition of the present disclosure.

In some aspects, the present disclosure provides a method of treating an Andes hantavirus (ANDV) infection in a subject in need thereof, comprising administering to the subject a therapeutically effective amount of a combination product of the present disclosure or a pharmaceutical composition of the present disclosure.

In some aspects, the present disclosure provides a method of treating a Sin Nombre Hantavirus (SNV) infection in a subject in need thereof, comprising administering to the subject a therapeutically effective amount of a combination product of the present disclosure or a pharmaceutical composition of the present disclosure.

In some aspects, the present disclosure provides a method of treating a Four Corners hantavirus (SNV) infection in a subject in need thereof, comprising administering to the subject a therapeutically effective amount of a combination product of the present disclosure or a pharmaceutical composition of the present disclosure.

In some aspects, the present disclosure provides a method of treating a Seoul virus (SEOV) infection in a subject in need thereof, comprising administering to the subject a therapeutically effective amount of a combination product of the present disclosure or a pharmaceutical composition of the present disclosure.

In some aspects, the present disclosure provides a method of treating a Laguna Negra virus (LANV) infection in a subject in need thereof, comprising administering to the subject a therapeutically effective amount of a combination product of the present disclosure or a pharmaceutical composition of the present disclosure.

In some aspects, the present disclosure provides a method of treating an Araraquara virus infection in a subject in need thereof, comprising administering to the subject a therapeutically effective amount of a combination product of the present disclosure or a pharmaceutical composition of the present disclosure.

In some aspects, the present disclosure provides a method of treating a Juquitiba virus infection in a subject in need thereof, comprising administering to the subject a therapeutically effective amount of a combination product of the present disclosure or a pharmaceutical composition of the present disclosure.

In some aspects, the present disclosure provides a method of treating a Dobrava virus (DOBV) infection in a subject in need thereof, comprising administering to the subject a therapeutically effective amount of a combination product of the present disclosure or a pharmaceutical composition of the present disclosure.

In some aspects, the present disclosure provides a method of treating a Puumala virus (PUUV) infection in a subject in need thereof, comprising administering to the subject a therapeutically effective amount of a combination product of the present disclosure or a pharmaceutical composition of the present disclosure.

In some aspects, the present disclosure provides a method of treating a Black Creek Canal virus infection in a subject in need thereof, comprising administering to the subject a therapeutically effective amount of a combination product of the present disclosure or a pharmaceutical composition of the present disclosure.

In some aspects, the present disclosure provides a method of treating a Choclo virus infection in a subject in need thereof, comprising administering to the subject a therapeutically effective amount of a combination product of the present disclosure or a pharmaceutical composition of the present disclosure.

In some aspects, the present disclosure provides a method of treating a Lechiguanas virus infection in a subject in need thereof, comprising administering to the subject a therapeutically effective amount of a combination product of the present disclosure or a pharmaceutical composition of the present disclosure.

In some aspects, the present disclosure provides a method of treating a Monongahela virus infection in a subject in need thereof, comprising administering to the subject a therapeutically effective amount of a combination product of the present disclosure or a pharmaceutical composition of the present disclosure.

In some aspects, the present disclosure provides a method of treating a New York virus infection in a subject in need thereof, comprising administering to the subject a therapeutically effective amount of a combination product of the present disclosure or a pharmaceutical composition of the present disclosure.

In some aspects, the present disclosure provides a method of treating Tula virus in a subject in need thereof, comprising administering to the subject a therapeutically effective amount of a combination product of the present disclosure or a pharmaceutical composition of the present disclosure.

In some aspects, the present disclosure provides a method of treating an Amur virus infection in a subject in need thereof, comprising administering to the subject a therapeutically effective amount of a combination product of the present disclosure or a pharmaceutical composition of the present disclosure.

In some aspects, the present disclosure provides a method of treating a Sangassou virus infection in a subject in need thereof, comprising administering to the subject a therapeutically effective amount of a combination product of the present disclosure or a pharmaceutical composition of the present disclosure.

In some aspects, the present disclosure provides a method of treating a Thottapalayam virus (TPMV) infection in a subject in need thereof, comprising administering to the subject a therapeutically effective amount of a combination product of the present disclosure or a pharmaceutical composition of the present disclosure.

In some aspects, the present disclosure provides a method of treating an Oxbow virus infection in a subject in need thereof, comprising administering to the subject a therapeutically effective amount of a combination product of the present disclosure or a pharmaceutical composition of the present disclosure.

In some aspects, the present disclosure provides a method of treating a Luxi virus infection in a subject in need thereof, comprising administering to the subject a therapeutically effective amount of a combination product of the present disclosure or a pharmaceutical composition of the present disclosure.

In some aspects, the present disclosure provides a combination product of the present disclosure for use in inhibiting of virus RNA replication (e.g., in vitro or in vivo).

In some aspects, the present disclosure provides a combination product of the present disclosure for use in treating or preventing a disease or disorder disclosed herein.

In some aspects, the present disclosure provides a combination product of the present disclosure for use in treating or preventing a viral infection disclosed herein.

In some aspects, the present disclosure provides a combination product of the present disclosure for use in treating a disease or disorder disclosed herein.

In some aspects, the present disclosure provides a combination product of the present disclosure for use in treating a viral infection disclosed herein.

In some aspects, the present disclosure provides a combination product of the present disclosure for use in treating or preventing an Andes hantavirus (ANDV) infection in a subject in need thereof.

In some aspects, the present disclosure provides a combination product of the present disclosure for use in treating or preventing a Sin Nombre Hantavirus (SNV) infection in a subject in need thereof.

In some aspects, the present disclosure provides a combination product of the present disclosure for use in treating or preventing a Four Corners hantavirus (SNV) infection in a subject in need thereof.

In some aspects, the present disclosure provides a combination product of the present disclosure for use in treating or preventing a Seoul virus (SEOV) infection in a subject in need thereof.

In some aspects, the present disclosure provides a combination product of the present disclosure for use in treating or preventing a Laguna Negra virus (LANV) infection in a subject in need thereof.

In some aspects, the present disclosure provides a combination product of the present disclosure for use in treating or preventing an Araraquara virus infection in a subject in need thereof.

In some aspects, the present disclosure provides a combination product of the present disclosure for use in treating or preventing a Juquitiba virus infection in a subject in need thereof.

In some aspects, the present disclosure provides a combination product of the present disclosure for use in treating or preventing a Dobrava virus (DOBV) infection in a subject in need thereof.

In some aspects, the present disclosure provides a combination product of the present disclosure for use in treating or preventing a Puumala virus (PUUV) infection in a subject in need thereof.

In some aspects, the present disclosure provides a combination product of the present disclosure for use in treating or preventing a Black Creek Canal virus infection in a subject in need thereof.

In some aspects, the present disclosure provides a combination product of the present disclosure for use in treating or preventing a Choclo virus infection in a subject in need thereof.

In some aspects, the present disclosure provides a combination product of the present disclosure for use in treating or preventing a Lechiguanas virus infection in a subject in need thereof.

In some aspects, the present disclosure provides a combination product of the present disclosure for use in treating or preventing a Monongahela virus infection in a subject in need thereof.

In some aspects, the present disclosure provides a combination product of the present disclosure for use in treating or preventing a New York virus infection in a subject in need thereof.

In some aspects, the present disclosure provides a combination product of the present disclosure for use in treating or preventing a Tula virus infection in a subject in need thereof.

In some aspects, the present disclosure provides a combination product of the present disclosure for use in treating or preventing an Amur virus infection in a subject in need thereof.

In some aspects, the present disclosure provides a combination product of the present disclosure for use in treating or preventing a Sangassou virus infection in a subject in need thereof.

In some aspects, the present disclosure provides a combination product of the present disclosure for use in treating or preventing a Thottapalayam virus (TPMV) infection in a subject in need thereof.

In some aspects, the present disclosure provides a combination product of the present disclosure for use in treating or preventing an Oxbow virus infection in a subject in need thereof.

In some aspects, the present disclosure provides a combination product of the present disclosure for use in treating or preventing a Luxi virus infection in a subject in need thereof.

In some aspects, the present disclosure provides a combination product of the present disclosure for use in treating an Andes hantavirus (ANDV) infection in a subject in need thereof.

In some aspects, the present disclosure provides a combination product of the present disclosure for use in treating a Sin Nombre Hantavirus (SNV) infection in a subject in need thereof.

In some aspects, the present disclosure provides a combination product of the present disclosure for use in treating a Four Corners hantavirus (SNV) infection in a subject in need thereof.

In some aspects, the present disclosure provides a combination product of the present disclosure for use in treating a Seoul virus (SEOV) infection in a subject in need thereof.

In some aspects, the present disclosure provides a combination product of the present disclosure for use in treating a Laguna Negra virus (LANV) infection in a subject in need thereof.

In some aspects, the present disclosure provides a combination product of the present disclosure for use in treating an Araraquara virus infection in a subject in need thereof.

In some aspects, the present disclosure provides a combination product of the present disclosure for use in treating a Juquitiba virus infection in a subject in need thereof.

In some aspects, the present disclosure provides a combination product of the present disclosure for use in treating a Dobrava virus (DOBV) infection in a subject in need thereof.

In some aspects, the present disclosure provides a combination product of the present disclosure for use in treating a Puumala virus (PUUV) infection in a subject in need thereof.

In some aspects, the present disclosure provides a combination product of the present disclosure for use in treating a Black Creek Canal virus infection in a subject in need thereof.

In some aspects, the present disclosure provides a combination product of the present disclosure for use in treating a Choclo virus infection in a subject in need thereof.

In some aspects, the present disclosure provides a combination product of the present disclosure for use in treating a Lechiguanas virus infection in a subject in need thereof.

In some aspects, the present disclosure provides a combination product of the present disclosure for use in treating a Monongahela virus infection in a subject in need thereof.

In some aspects, the present disclosure provides a combination product of the present disclosure for use in treating a New York virus infection in a subject in need thereof.

In some aspects, the present disclosure provides a combination product of the present disclosure for use in treating a Tula virus infection in a subject in need thereof.

In some aspects, the present disclosure provides a combination product of the present disclosure for use in treating an Amur virus infection in a subject in need thereof.

In some aspects, the present disclosure provides a combination product of the present disclosure for use in treating a Sangassou virus infection in a subject in need thereof.

In some aspects, the present disclosure provides a combination product of the present disclosure for use in treating a Thottapalayam virus (TPMV) infection in a subject in need thereof.

In some aspects, the present disclosure provides a combination product of the present disclosure for use in treating an Oxbow virus infection in a subject in need thereof.

In some aspects, the present disclosure provides a combination product of the present disclosure for use in treating a Luxi virus infection in a subject in need thereof.

In some aspects, the present disclosure provides use of a combination product of the present disclosure in the manufacture of a medicament for inhibiting viral RNA replication (e.g., in vitro or in vivo).

In some aspects, the present disclosure provides use of a combination product of the present disclosure or a pharmaceutical composition thereof in the manufacture of a medicament for treating or preventing a disease or disorder disclosed herein.

In some aspects, the present disclosure provides use of a pharmaceutical composition of the present disclosure in the manufacture of a medicament for treating or preventing a disease or disorder disclosed herein.

In some aspects, the present disclosure provides use of a combination product of the present disclosure in the manufacture of a medicament for treating or preventing a viral infection disclosed herein.

In some aspects, the present disclosure provides use of a combination product of the present disclosure or a pharmaceutical composition thereof in the manufacture of a medicament for treating a disease or disorder disclosed herein.

In some aspects, the present disclosure provides use of a combination product of the present disclosure in the manufacture of a medicament for treating a viral infection disclosed herein.

In some aspects, the present disclosure provides use of a combination product of the present disclosure in the manufacture of a medicament for treating an Orthohantavirus infection disclosed herein.

In some aspects, the present disclosure provides use of a pharmaceutical composition of the present disclosure in the manufacture of a medicament for treating a viral infection disclosed herein.

In some aspects, the present disclosure provides use of a pharmaceutical composition of the present disclosure in the manufacture of a medicament for treating an Orthohantavirus infection disclosed herein.

In some aspects, the present disclosure provides use of a combination product of the present disclosure in the manufacture of a medicament for treating or preventing an Andes hantavirus (ANDV) infection in a subject in need thereof.

In some aspects, the present disclosure provides use of a combination product of the present disclosure in the manufacture of a medicament for treating or preventing a Sin Nombre Hantavirus (SNV) infection in a subject in need thereof.

In some aspects, the present disclosure provides use of a combination product of the present disclosure in the manufacture of a medicament for treating or preventing a Four Corners hantavirus (SNV) infection in a subject in need thereof.

In some aspects, the present disclosure provides use of a combination product of the present disclosure in the manufacture of a medicament for treating or preventing a Seoul virus (SEOV) infection in a subject in need thereof.

In some aspects, the present disclosure provides use of a combination product of the present disclosure in the manufacture of a medicament for treating or preventing a Laguna Negra virus (LANV) infection in a subject in need thereof.

In some aspects, the present disclosure provides use of a combination product of the present disclosure in the manufacture of a medicament for treating or preventing an Araraquara virus infection in a subject in need thereof.

In some aspects, the present disclosure provides use of a combination product of the present disclosure in the manufacture of a medicament for treating or preventing a Juquitiba virus infection in a subject in need thereof.

In some aspects, the present disclosure provides use of a combination product of the present disclosure in the manufacture of a medicament for treating or preventing a Dobrava virus (DOBV) infection in a subject in need thereof.

In some aspects, the present disclosure provides use of a combination product of the present disclosure in the manufacture of a medicament for treating or preventing a Puumala virus (PUUV) infection in a subject in need thereof.

In some aspects, the present disclosure provides use of a combination product of the present disclosure in the manufacture of a medicament for treating or preventing a Black Creek Canal virus infection in a subject in need thereof.

In some aspects, the present disclosure provides use of a combination product of the present disclosure in the manufacture of a medicament for treating or preventing a Choclo virus infection in a subject in need thereof.

In some aspects, the present disclosure provides use of a combination product of the present disclosure in the manufacture of a medicament for treating or preventing a Lechiguanas virus infection in a subject in need thereof.

In some aspects, the present disclosure provides use of a combination product of the present disclosure in the manufacture of a medicament for treating or preventing a Monongahela virus infection in a subject in need thereof.

In some aspects, the present disclosure provides use of a combination product of the present disclosure in the manufacture of a medicament for treating or preventing a New York virus infection in a subject in need thereof.

In some aspects, the present disclosure provides use of a combination product of the present disclosure in the manufacture of a medicament for treating or preventing a Tula virus infection in a subject in need thereof.

In some aspects, the present disclosure provides use of a combination product of the present disclosure in the manufacture of a medicament for treating or preventing an Amur virus infection in a subject in need thereof.

In some aspects, the present disclosure provides use of a combination product of the present disclosure in the manufacture of a medicament for treating or preventing a Sangassou virus infection in a subject in need thereof.

In some aspects, the present disclosure provides use of a combination product of the present disclosure in the manufacture of a medicament for treating or preventing a Thottapalayam virus (TPMV) infection in a subject in need thereof.

In some aspects, the present disclosure provides use of a combination product of the present disclosure in the manufacture of a medicament for treating or preventing an Oxbow virus infection in a subject in need thereof.

In some aspects, the present disclosure provides use of a combination product of the present disclosure in the manufacture of a medicament for treating or preventing a Luxi virus infection in a subject in need thereof.

The present disclosure provides combination products and pharmaceutical compositions that function as inhibitors of Orthohantavirus replication (e.g., in vitro or in vivo).

The present disclosure therefore provides a method of inhibiting of Orthohantavirus replication in vitro or in vivo, said method comprising contacting a cell with a therapeutically effective amount of a compound, or a pharmaceutically acceptable salt thereof, as defined herein.

In some embodiments, the inhibitor of Orthohantavirus RNA replication is a combination product of the present disclosure.

In some embodiments, the inhibitor of Orthohantavirus RNA replication is a pharmaceutical composition of the present disclosure.

Industry-accepted assays/disease models can determine effectiveness of combination products of the disclosure according to standard practices of elucidating the same as described in the art and are found in the current general knowledge.

The present disclosure also provides a method of treating a viral infection in a subject in need of such treatment, said method comprising administering to said subject a therapeutically effective amount of a combination product or a pharmaceutical composition as defined herein.

Another aspect of the invention relates to use of combination of (i) favipiravir and (ii) mycophenolate for treating diseases caused by RNA virus infection including Andes hantavirus (ANDV), Sin Nombre Hantavirus (SNV), Four Corners hantavirus (SNV), Seoul virus (SEOV), Laguna Negra virus (LANV), Araraquara virus, Juquitiba virus, Dobrava virus (DOBV), Puumala virus (PUUV), Black Creek Canal virus, Choclo virus, Lechiguanas virus, Monongahela virus, New York virus, Tula virus, Amur virus, Sangassou virus, Thottapalayam virus (TPMV), Oxbow virus, and Luxi virus.

In some embodiments, the subject is a mammal.

In some embodiments, the subject is a human.

### Routes of Administration

The combination products of the disclosure or pharmaceutical compositions comprising these combination products may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Examples of Combination Products

In the Table 2 presented certain non-limiting examples of the combination products.

TABLE 2
Selected examples of the combination products

| | Inhibitor of viral RNA | | Molar | Dose, |
|---|---|---|---|---|
| # | replication | IMPDH inhibitor | ratio | mg/kg |
| 1 | favipiravir | mycophenolate mofetil | 1:1 | 0.3 |
| 2 | favipiravir | mycophenolate mofetil | 1:2 | 0.6 |
| 3 | favipiravir | mycophenolate mofetil | 1:5 | 1.5 |
| 4 | favipiravir | mycophenolate mofetil | 1:10 | 3 |
| 5 | favipiravir | mycophenolate mofetil | 1:50 | 15 |
| 6 | favipiravir | mycophenolate mofetil | 1:100 | 30 |
| 7 | favipiravir | mycophenolate mofetil | 2:1 | 0.6 |
| 8 | favipiravir | mycophenolate mofetil | 5:1 | 1.5 |
| 9 | favipiravir | mycophenolate mofetil | 10:1 | 3 |
| 10 | favipiravir | mycophenolate mofetil | 50:1 | 15 |
| 11 | favipiravir | mycophenolate mofetil | 100:1 | 30 |
| 12 | favipiravir-NTP | mycophenolate mofetil | 1:1 | 0.3 |
| 13 | favipiravir-NTP | mycophenolate mofetil | 1:2 | 0.6 |
| 14 | favipiravir-NTP | mycophenolate mofetil | 1:5 | 1.5 |
| 15 | favipiravir-NTP | mycophenolate mofetil | 1:10 | 3 |
| 16 | favipiravir-NTP | mycophenolate mofetil | 1:50 | 15 |
| 17 | favipiravir-NTP | mycophenolate mofetil | 1:100 | 30 |
| 18 | favipiravir-NTP | mycophenolate mofetil | 2:1 | 0.6 |
| 19 | favipiravir-NTP | mycophenolate mofetil | 5:1 | 1.5 |
| 20 | favipiravir-NTP | mycophenolate mofetil | 10:1 | 3 |
| 21 | favipiravir-NTP | mycophenolate mofetil | 50:1 | 15 |
| 22 | favipiravir-NTP | mycophenolate mofetil | 100:1 | 30 |
| 23 | favipiravir | mycophenolic acid | 1:1 | 0.3 |
| 24 | favipiravir | mycophenolic acid | 1:2 | 0.6 |
| 25 | favipiravir | mycophenolic acid | 1:5 | 1.5 |
| 26 | favipiravir | mycophenolic acid | 1:10 | 3 |
| 27 | favipiravir | mycophenolic acid | 1:50 | 15 |
| 28 | favipiravir | mycophenolic acid | 1:100 | 30 |
| 29 | favipiravir | mycophenolic acid | 2:1 | 0.6 |
| 30 | favipiravir | mycophenolic acid | 5:1 | 1.5 |
| 31 | favipiravir | mycophenolic acid | 10:1 | 3 |
| 32 | favipiravir | mycophenolic acid | 50:1 | 15 |
| 33 | favipiravir | mycophenolic acid | 100:1 | 30 |
| 34 | favipiravir-NTP | mycophenolic acid | 1:1 | 0.3 |
| 35 | favipiravir-NTP | mycophenolic acid | 1:2 | 0.6 |
| 36 | favipiravir-NTP | mycophenolic acid | 1:5 | 1.5 |
| 37 | favipiravir-NTP | mycophenolic acid | 1:10 | 3 |
| 38 | favipiravir-NTP | mycophenolic acid | 1:50 | 15 |
| 39 | favipiravir-NTP | mycophenolic acid | 1:100 | 30 |
| 40 | favipiravir-NTP | mycophenolic acid | 2:1 | 0.6 |
| 41 | favipiravir-NTP | mycophenolic acid | 5:1 | 1.5 |
| 42 | favipiravir-NTP | mycophenolic acid | 10:1 | 3 |
| 43 | favipiravir-NTP | mycophenolic acid | 50:1 | 15 |
| 44 | favipiravir-NTP | mycophenolic acid | 100:1 | 30 |

### Examples of Pharmaceutical Compositions

Certain non-limiting examples of the pharmaceutical compositions of the combination products described here

The pharmaceutical composition according to disclosure comprises combination product and auxiliary compounds.

The examples of combination products according to invention presented in the Table 2.

According to an embodiment of the present invention, the pharmaceutical composition may be in the form of tablets, capsules, tablets filled in capsule, minitablets filled in capsule, sachets containing powder or granules, pellets, and the like. The pharmaceutical composition is meant for once daily or twice daily administration.

The preparation of film coated tablets having some of the auxiliary ingredients as listed below which can either include or exclude when prepared using conventional techniques:
Sodium lauryl sulfate
Microcrystalline cellulose
Lactose monohydrate
Lactose 200 mesh
Croscarmellose sodium
Hydroxypropyl methyl cellulose (Hypromellose)
Sodium carboxymethylcellulose
Talc
Magnesium stearate
Lactose DC
Maize starch
Pregelatinized starch
Colloidal silicon dioxide
Polyethylene glycol 6000 (macrogol)
Basic calcium phosphate
Menitol
Ethyl cellulose
Hydroxypropyl methylcellulose k100
Light magnesium oxide
Magnesium hydroxide
Calcium CMC
Hydrogenated castor oil
Sodium starch glycolate
Sodium benzoate
Sodium methyl parabe sodium propyl paraben
PVP k30
PVP k90
FILM COAT
Titanium dioxide
Polyethylene glycol 6000 (macrogol).

### Biological Assays

### Example A. Inhibition Test for Flu Virus (and Other Respiratory Viruses)

MDCK cell expansion: Mardin-Darbin canine kidney cells were maintained in modified Eagle's medium (MEM) containing 10% of fetal bovine serum (FBS). When the confluent monolayer reached 80 to 90%, cells were washed twice with phosphate buffer solution (PBS) and treated with trypsin for 14 minutes. Then, MDCK cell were resuspended in 10 ml of MEM 10% media and counted. For new flask passages, the cells were diluted 1 in 3 and seeded in 15 ml of media. For inhibition assays, MDCK cells were seed at 2×10⁴ MDCK cells/well in a 96 well plate. In both cases, cells were incubated at 37 °C and 5% CO₂.

Note: MDCK cells should be in low passages and test negative for mycoplasma and other pathogens before use. For other respiratory viruses testing cells, time, and virus concentration varies.

Flu virus stock preparation: 5×10⁵ MDCK cells were seeded in T75 flask, after 24 hours of incubation at 37 °C and 5% CO₂, cells were washed twice and inoculated with Flu virus strain A/Puerto Rico/8/1934 (PR8) at 0.01 multiplicity of infection (MOI) for 1 hour at 37 °C and 5% CO₂. After incubation, 10 ml of MEM 0.35% of Bovine serum albumin (BSA), 1 μg/ml TPCK-trypsin was added. Supernatant were collected at 24, 48, and 72 hours post infection, aliquoted and kept at −80 °C for viral titration.

Viral titration by plaque forming units: To determine the Plaque formation units (PFU), 10-fold viral dilutions were prepared from the viral stocks. MDCK cells were seeded in 6 well plates. 24 hours later, when the monolayer reached between 80 and 90%, cells were washed twice with PBS and inoculated with viral stock dilutions. Incubated for one hour and cover with semisolid media containing 10 ml of MEM 0.35% of BSA, 1 μg/ml TPCK-trypsin, and 0.3% agarose. After 72 hours incubation at 37 °C and 5% CO₂, all plaques were stained with crystal violet at described below. The PFU were determined by counting plaques in the dilution producing around 50 visible plaques and then applying the following formula: PFU/ml=(N×10^{X})/V where N is the number of plaques, X is the dilution counted, and V is the virus volume used per well.

Antiviral compounds: Favipiravir and Mycophenolate were prepared as 10 mM stocks in dimethyl sulfoxide (DMSO) and were soluble when diluted in various reaction mixtures and cell culture media. Favipiravir was tested using concentrations ranging from 200 μM to 1.56 μM, with an estimated IC₅₀ of 25 μM when used alone. Mycophenolate was tested in concentrations between 2 μM and 0.25 μM with an estimated IC₅₀ of 1.422 μM when used alone. Also, combination of Favipiravir with Mycophenolate was tested.

MDCK viral infection: After 24 hours, those cells in 96 well plates were wash twice with PBS and infected with PR8 Flu virus at 0.01 multiplicity of infection MOI for 1 hour at 37 °C and 5% CO₂. After incubation, the inoculum was carefully aspirated and replaced with MEM 0.35% of Bovine serum albumin (BSA), 1 μg/ml TPCK-trypsin, and different concentration of the antiviral drug.

Crystal violet staining: After 48 hours of incubation, the media was aspirated and MDCK cells washed once with PBS. The infectious virus was inactivated by filling each well with 4% paraformaldehyde and incubating 20 minutes at room temperature (rt). Then, the liquid was aspirated, and the wells were filled with crystal violet. After 10 minutes of incubation at RT, the crystal violet was carefully washed with tap water and the plates were dried overnight. At this point, the EC₅₀ could be visually estimated.

OD readings: Dried plate were treated with 1% SDS to solubilize the stain. This process took 20 minutes with constant shaking until the color was uniform. Then, the optical densities (OD) of each well were measured at 590 nm.

IC₅₀ calculation: To calculate the IC₅₀ concentrations, the log of the μM drug concentrations was compared with the % of inhibition based on the OD readings using excel software. IC₅₀ was calculated using a four-parameter logistic nonlinear regression model by GraphPad Prism.

Synergistic interaction analysis: The SynergyFinder2.0 was used to calculate the highest single agent (HSA) synergy score of two-drug combinations from different pairwise combinations. Areas with synergy score less than −10: the interaction between two drugs is likely to be antagonistic; from −10 to 10: the interaction between two drugs is likely to be additive; larger than 10: the interaction between two drugs is likely to be synergistic.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A method of inhibiting of RNA replication of Orthohantavirus, comprising of administering to a subject a combination product comprising (i) favipiravir and (ii) mycophenolate.

2. The method of claim 1, wherein favipiravir is selected from: nucleoside base, favipiravir ribonucleotide, or favipiravir nucleotide triphosphate: 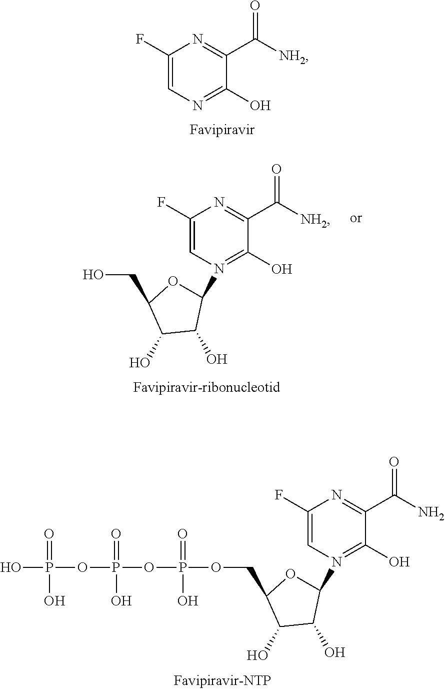
or a pharmaceutically acceptable salt, solvate, or tautomer thereof.

3. The method of claim 1, wherein mycophenolate is selected from: mycophenolic acid, sodium salt of mycophenolic acid, or mycophenolate mofetil: 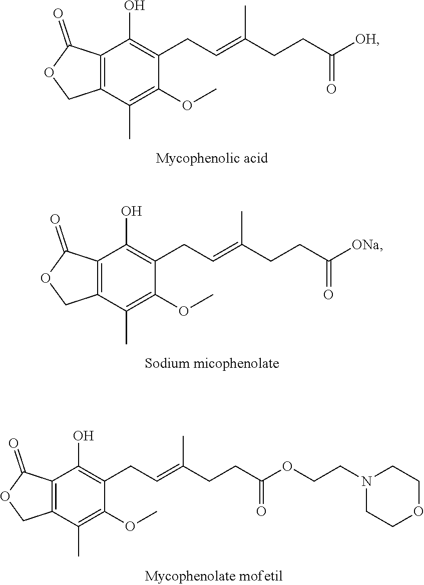
or solvate, or tautomer thereof.

4. A method of treating a viral infection caused by Orthohantavirus, comprising of administering to a subject a combination product comprising (i) favipiravir and (ii) mycophenolate.

5. The method of claim 4, wherein favipiravir is selected from: nucleoside base, favipiravir ribonucleotide, or favipiravir nucleotide triphosphate: 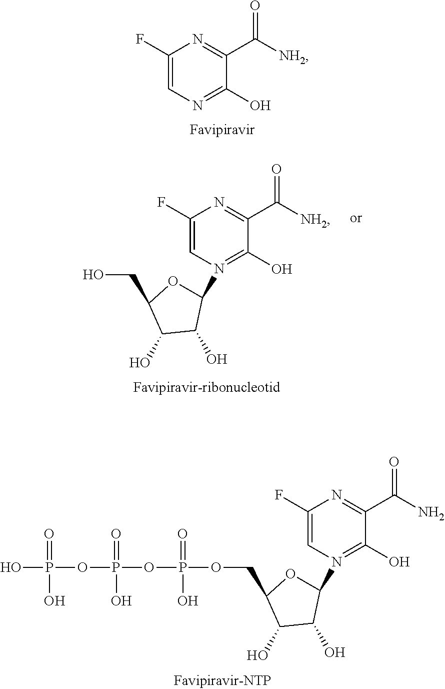
or a pharmaceutically acceptable salt, solvate, or tautomer thereof.

6. The method of claim 4, wherein mycophenolate is selected from: mycophenolic acid, sodium salt of mycophenolic acid, or mycophenolate mofetil: 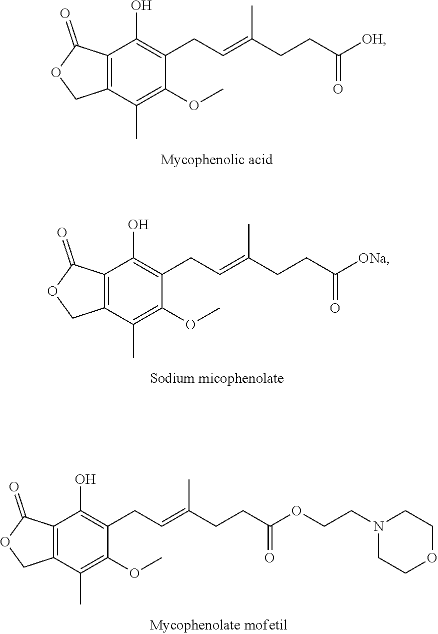
or solvate, or tautomer thereof.

7. The method of claim 4 wherein virus selected from Andes hantavirus (ANDV), Sin Nombre Hantavirus (SNV), Four Corners hantavirus (SNV), Seoul virus (SEOV), Laguna Negra virus (LANV), Araraquara virus, Juquitiba virus, Dobrava virus (DOBV), Puumala virus (PUUV), Black Creek Canal virus, Choclo virus, Lechiguanas virus, Monongahela virus, New York virus, Tula virus, Amur virus, Sangassou virus, Thottapalayam virus (TPMV), Oxbow virus, and Luxi virus.

8. The method of any one of claims 1-7, wherein the subject is a mammal.

9. The method of claim 8, wherein the subject is a human.
